(19) United States
(12) Patent Application Publication
(10) Pub. No.: US 2024/0093261 A1
(43) Pub. Date: Mar. 21, 2024

(54) HIGH PERFORMANCE COMPUTATION USING MICROORGANISMS

(71) Applicant: BACTERIA INTELLIGENCE LTD, London (GB)

(72) Inventors: Francesco GARDIN, Rivigo (IT); Rita PIZZI, Pizzighettone (IT)

(21) Appl. No.: 18/264,842

(22) PCT Filed: Feb. 9, 2022
(86) PCT No.: PCT/GB2022/050352

§ 371 Date: Aug. 9, 2023

(30) Foreign Application Priority Data
Feb. 10, 2021 (GB) 2101850.2

(51) Int. Cl. C12Q 1/02; G06N 3/00

(52) U.S. Cl. CPC C12Q 1/02; G06N 3/002

(57) ABSTRACT

The invention provides apparatus for implementing a computational system comprising: a colony of live microorganisms and a receptacle in which the microorganisms are able to be stimulated to cause a reaction or to modify their physiology or behaviour. Monitoring apparatus tracks the reaction or changes in the physiology or the behaviour of the microorganisms in response to stimulation; and processing circuitry makes calculations, including machine learning, based on or coupled with the reaction of the microorganisms, or their physiological or behavioural changes. The invention also proved a method of implementing a computational system comprising stimulating a colony of live microorganisms with a stimulus whose pattern is derived from input data, or allowing the stimulation with an external stimulus. The reaction or modification is monitored and output data based on the reaction or modification is derived.

The present invention provides apparatus for implementing a high performance computational system using microorganisms. A specific example includes an artificial neural network (ANN) using a hybrid digital-biological system. It further relates to a method of implementing computational system and to software for performing at least part of that method using a hardware-biological interface.

In the literature there are known attempts to make a particular organism, the slime mould, perform computational processes (“Logical Gates and Circuits Implemented in Slime Mould”, Advances in Physarum Machines. Sensing and Computing with Slime Mould, A. Adamatzky, et al Springer, 2016; and “Twenty five uses of slime mould in electronics and computing”: Survey, Int. Journ. of Unconventional Computing, Vol. 11, pp. 449-471 A. Adamatzky Old City Publishing) and the exploitation for educational and entertainment purposes of the phototactical behaviour of Euglenas (P. Washington, et al: A cloud-based development toolkit for programming experiments and interactive applications with living cells, bioRxiv 236919; and H. Kim, et al, LudusScope: Accessible Interactive Smartphone Microscopy for Life-Science Education, PLOS ONE 0162602). The slime mould exhibits interesting behaviours but is extremely slow in its movements and therefore unsuitable for real-world applications competitive with a computer. The above attempts with Euglenas, although they show cyanobacteria phototaxis as a promising function, have not produced possible applications because they lack a mechanism for functionality and a computational model of reference.

The present invention provides a system that can be miniaturised and which performs advanced problem-solving and computational functions. It is based on a synergy between hardware, software and biological systems. The system exploits the tactic response of microorganism (such as bacteria and flagellates) and the self-organising capacity of complex systems, particularly biological systems. The system can perform analyses directly interfaced with biological organisms, or it can be useful if applied to industrial systems, including robotic systems, which require miniaturisation of computational processes.

The invention is based on the use of stimulation (e.g. light stimulation) and corresponding microorganism reactions analysed by means of a coupled computational model. The invention therefore solves the problem of performing complex computations directly onboard in a particularly rapid and miniaturised way.

The present invention may in a broad aspect of certain embodiments consist of a microorganism substrate and a computational system, which includes machine learning (ML) methods, that interprets the reactions or modifications of the microorganisms, such as cyanobacteria, bacteria, viruses, archaea, protists, and unicellular fungi, among others, and allows useful outputs to be drawn from their collective behaviour, to use them as a computational tool.

In some such embodiments the machine learning method developed exploits the functional isomorphism between an ANN, the Hopfield network, and the QPSO algorithm, Quantum Particle Swarm Optimization, which, like the Hopfield network, looks for the global minimum of a dynamic system by finding the minimum energy points where the system stabilises.

The Hopfield network is useful for our purpose because it allows a simple input of patterns in the form of a bitmap representing the data to be worked on. The QPSO algorithm instead calculates the global minimum of a function and finds the solution to the problem by following the path of a swarm of particles, and calculates from their movements the parameters useful for the evaluation of the global minimum.

ML methods are developing rapidly for problem solving in every field of application (pattern recognition, numeric calculus, forecasting, etc.), but require an intense computational load that is often delegated to powerful computers. This invention aims to delegate the computational load to colonies of microorganism with obvious economic and size advantages.

According to a first aspect of the present invention there is provided apparatus for implementing a computational system comprising:
a colony of live microorganisms;
a receptacle in which the microorganisms are able to be stimulated to cause a reaction or to modify their physiology or behaviour;
monitoring apparatus to track the reaction or changes in the physiology or the behaviour of the microorganisms in response to stimulation; and
processing circuitry to make calculations, including machine learning, based on or coupled with the reaction of the microorganisms, or their physiological or behavioural changes.

The nature of the reaction or modification in the physiology or behaviour of the microorganism can vary considerably. This will depend on the nature of the microorganism, and the nature of the stimulation. The monitoring apparatus will also need to be able to detect the reaction or modification so they should be paired accordingly. For example, a camera is suited to detect visible changes such as movement.

The reactions and modification/changes can be temporary or lasting in nature. For example, a reaction may simply be a movement from one position to another. Alternatively, a modification may be more lasting in effect, such as a lasting change in behaviour caused by the stimulus. Changes may include but are not limited to movement (i.e. changes in positions), changes in orientation, changes in shape, changes in electrical signal, genomic alterations, and changes in internal structure (e.g. organelle shape, number, position).

The response and sensitivity of the microorganisms varies according to the species. The microorganism needs sufficiently and quickly enough to respond to whatever stimuli is applied or encountered, which may vary depending on the nature of the stimulus and the application. The speed and nature of reaction or physiological response is important again dependent on the implementation.

Ideally, the microorganisms are cyanobacteria, bacteria, viruses, archaea, protists, and unicellular fungi. Cultures of the genera Euglena and Chlamydomonas are particularly good, and Euglena gracilis, has been found to be very effective in responding to light stimuli. Different organisms may be appropriate for reaction to other stimuli.

In one embodiment the invention relies on the reaction of the microorganisms in response to stimuli. Various forms of modification of physiology and/or behaviour of microorganisms, like taxis and associated stimuli may be appropriate. These may include phototaxis (stimulation by light), aerotaxis (stimulation by oxygen), anemotaxis (stimulation by wind), barotaxis (stimulation by pressure), chemotaxis (stimulation by chemicals); durotaxis (stimulation by stiffness), electrotaxis or galvanotaxis (stimulation by electric current); gravitaxis (stimulation by gravity), hydrotaxis (stimulation by moisture); magnetotaxis (stimulation by magnetic field), rheotaxis (stimulation by fluid flow), thermotaxis (stimulation by changes in temperature), thigmotaxis (by physical contact).

Stimulation of the microorganisms can be by stimuli that are controlled and/or delivered by a part of the apparatus. In this context the apparatus may further include stimulation apparatus to stimulate the microorganisms within the receptacle. Such stimulation may be derived from input data. This allows specific input data to be applied to see the reaction of the microorganisms. Alternatively, the stimuli may be derived externally of the apparatus of the present invention. In this context the stimulus may come from environmental factors or from things that the receptacle is brought into contact with. In such a scenario an output derived from the reaction of the microorganisms can be used to assess the nature of the input stimuli and make a judgement thereon. That could be based on a comparison to reactions to known stimuli or learning phases of stimulation.

The stimulus may be light, which may be visible light. If so, the stimulation apparatus may include one or more light emitter (e.g. LED, OLED, QLED, microLED, etc.) in a suitable array. For light stimulation, ideally the stimulation apparatus comprises a display screen. The screen may be of a size broadly comparable to the area of the receptacle. The stimulation apparatus may be located adjacent or close to the receptacle to ensure maximum effect of the light on the microorganisms. Ideally the screen is on one side of (e.g. below) the receptacle and the monitoring apparatus is on the opposed side. The receptacle is ideally at least partially translucent or transparent to the emitted light to ensure light reaches the microorganisms and so that the microorganisms are visible.

A light stimulus can vary, in one or more of the following characteristics: light intensity, wavelength, pattern and duration. The intensity relates to the brightness or luminescence of the light which may vary across a given image displayed. The wavelength relates to the colour or nature (e.g. visible/UV/infrared) of the light. It has been found that green light (wavelengths of about 560-520 nm) works well with Euglenas but suitable colour choice may vary dependent on the desired input stimulation. Pattern variation plays an important part in the present invention. An image as displayed to the microorganisms may vary in colour, brightness and shape/pattern. This variation is derived from varying input data and for desired output calculation to be done by the ANN. Altering the pattern plays a major role in that variation.

The stimulation apparatus may also permit or facilitate the direct interface with or coupling to another biological, natural, or artificial system to provide the stimulus. The microorganisms can be coupled with another biological system, with which it interacts: e.g. microorganisms in direct contact with a patient's skin, under a small petri dish can detect the presence of pathologies according to the same static of dynamic patterns observed in microorganisms when using other forms of stimulation. For example, a chemical sensor may analyse the sequence of chemical reactions generated by the interaction between the two biological systems: the microorganisms and the skin.

The apparatus may be provided with one or more input node and one or more output node. The reaction or physiological change of the microorganisms may be tracked relative to the input and output nodes. The stimulus pattern, such as an image displayed if light stimulation, may define the location of the one or more input nodes. The stimulus pattern may define the location of the one or more output nodes. One or more of the input and output nodes may be in the same location.

The receptacle needs to hold the microorganisms in such a way that they are able to respond to stimuli and remain viable for long enough to perform their function. The receptacle can be a petri-dish or other flat plate on which the microorganisms are mobile. The receptacle can be open topped or can be closed so that the microorganisms and whatever culture medium they are in remain contained regardless of orientation. The receptacle can be designed to create the best situation for the microorganism to exhibit problem solving. It can be fixed in shape or dynamic. It may have mazes, circuits and 2D or 3D environments. Indeed, changing the stimuli has the effect to dynamically vary the operational environment for the microorganisms.

The microorganisms are located in a receptacle and medium that maintains their viability and permits their reaction or change in response to stimulation as well as allowing monitoring of their reaction or change. A liquid culture medium as disclosed herein is suitable to provide the required growth nutrients.

As noted above the receptacle needs to permit stimulation and monitoring of the microorganisms therein. The configuration and design of the receptacle needs also to be appropriate for the location of use. It also needs to be appropriate for the type of stimulation.

The monitoring apparatus needs to be able to identify and monitor the microorganisms for their reaction or change. The nature of the change will govern the nature of the monitoring apparatus and what it aims to detect. The monitoring apparatus may include one or more of a microscope, camera and/or image processing software to track movement of the microorganisms. The same or alternative forms of monitoring apparatus may be suited to monitoring the reaction or physiological change of the microorganisms in response to other stimuli. For example if the reaction is electrical the monitoring apparatus needs to be capable of detecting electrical changes such as electrical charge, or changes in magnetic field strength.

The processing circuitry can employ various forms of machine learning. This may include one or more of various forms of artificial neural network, Hopfield network, particle swarm optimization, quantum particle swarm optimization, petri nets, planning trees, belief networks and/or decision trees.

The building blocks of the apparatus of the present invention may be considered in certain but not all embodiments based on light stimulation and movement tracking as the following:
A biological set consisting of a receptacle, such as a petri-dish, containing a culture of microorganisms.
A display to create a pattern of light for stimulation of the microorganisms, that display being appropriately located relative to the receptacle. Ideally the illumination is from side of, such as below, the receptacle.
An imaging system to visualise and track the microorganisms. This ideally is a microscope that frames the area in which the microorganisms move and sends the images via camera or equivalent image capture technology to a computer.
software that determines the optimal configuration for the light stimulation according to the problem to be solved, and software that processes the paths taken by the microorganisms and derives from those paths the solution to the problem.

According to a further aspect of the present invention there is provided a method of implementing a computational system comprising:
stimulating a colony of live microorganisms with a stimulus whose pattern or form is derived from input data, or allowing the stimulation of a colony of live microorganisms with an external stimulus;
monitoring the reaction or modification of the microorganisms in response to that stimulation; and
deriving output data based on the reaction or modification.

The method may be carried out in apparatus as disclosed herein. The calculation may be carried out in processing circuitry that makes calculations based on the reaction or modification of the microorganisms. The processing circuitry may be functioning using software as defined elsewhere in this application.

The nature of the stimulation may vary as discussed above. It may involve providing an input stimulation pattern to the microorganisms for a defined time period. If using light stimulation the stimulation may be achieved by a display screen located adjacent to the microorganisms. The display screen ideally shows an image/pattern derived from input data and configured to provide a stimulus that causes the microorganisms to react or be modified in a way to allow calculation of output data. The nature of the monitoring will vary depending on the change to be detected as is discussed elsewhere herein.

The calculations may be carried out on processing circuitry which generates the output data driven by the computations of the microorganisms, intrinsic in their reactions or behavioural/physiological changes.

The monitoring may vary depending on the nature of stimulus and/or reaction/modification. This may be continuous or may be at discrete points in a cycle of stimulation. It could be at one or more time point: prior to stimulation, during stimulation, or after stimulation. It may include visual location and tracking of the microorganisms at appropriate points.

The method may include a training phase during which output data derived from reaction or modifications of the microorganisms in response to known stimulations are used to improve the accuracy of output data in subsequent cycles of stimulation. Accuracy is improved by adjusting so that errors detected from any anticipated outcome are minimised or removed. The training phase is carried out in advance of a test/calculation cycle.

According to a still further aspect of the present invention there is provided a data processing system configured to implement a method as described herein, the system being configured to derive the stimulus from the input data and/or to calculate the output data based on the movement.

The data processing system can be provided separately from the stimulation apparatus, monitoring apparatus and receptacle but appropriately connected thereto. Alternatively, the data processing system can be integrated with the stimulation apparatus, monitoring apparatus and receptacle in a single module or other compact standalone form.

According to a yet further aspect of the present invention there is provided a computer program comprising computer software code for implementing a method as described herein, the code being configured to derive the stimulus from the input data and/or to calculate the output data based on the movement or physiological change when the program is run on a data processing system.

In some embodiments, the data processing system comprises, and/or is in communication with, one or more memories and/or memory devices that store the input data and/or output data as described herein and/or that store software code for performing a method as described herein. The data processing system may comprise, and/or may be in communication with, a computer system that generates, processes and/or provides the input data and/or that receives, processes and/or stores the output data. The input data and/or output data may be provided and/or received via a tangible, non-transitory medium, such as a computer readable medium. The input data and/or output data may also or instead be provided and/or received via an interface device, either over a tangible medium, including but not limited to optical or analogue communications lines, or intangibly using wireless techniques, including but not limited to microwave, infrared or other transmission techniques.

Embodiments can be implemented in any suitable data processing system, such as a suitably configured computer-based and/or processor-based system. The various functions of the data processing system described herein can be carried out in any desired and suitable manner. For example, the functions of the data processing system described herein can be implemented in hardware and/or software as desired. Thus, unless otherwise indicated, the various functional elements and means described herein may, for example, comprise a suitable processor or processors, controller or controllers, functional units, circuitry, processing logic, microprocessor arrangements, etc., that are operable to perform the various functions, etc., such as appropriately dedicated hardware elements (processing circuitry) and/or programmable hardware elements (processing circuitry) that can be programmed to operate in the desired manner.

The methods described herein may be implemented at least partially using software and/or computer programs. Thus, further embodiments comprise computer software specifically adapted to implement a method as described herein when installed on one or more data processors, a computer program element comprising computer software code portions for implementing a method as described herein when the program element is run on one or more data processors, and a computer program comprising code adapted to implement the steps of a method as described herein when the program is run on one or more data processors. Embodiments also extend to a computer software carrier comprising such software which, when used to operate a data processing system comprising one or more data processors, causes said system, in conjunction with said one or more data processors, to implement the steps of a method as described herein. Such a computer software carrier could be a physical storage medium, or could be a signal such as an electronic signal over wires, an optical signal or a radio signal such as to a satellite or the like.

It will further be appreciated that not all steps of the methods described herein need be implemented by computer software and thus further embodiments comprise computer software and such software installed on a computer software carrier for implementing at least one of the steps of a method as described herein.

Embodiments may accordingly comprise a computer program product for use with a computer system. Such an implementation may comprise a series of computer readable instructions for example fixed on a tangible, non-transitory medium, such as a computer readable medium. It could also comprise a series of computer readable instructions transmittable to a computer system, via an interface device, either over a tangible medium, including communications lines, or intangibly using wireless techniques. The series of computer readable instructions embodies all or part of the functionality described herein. Those skilled in the art will appreciate that such computer readable instructions can be written in a number of programming languages for use with many computer architectures or operating systems. Further, such instructions may be stored using any memory technology, present or future, including but not limited to, semiconductor, magnetic, or optical, or transmitted using any communications technology, present or future, including but not limited to optical, infrared, or microwave. It is contemplated that such a computer program product may be distributed as a removable medium with accompanying printed or electronic documentation, pre-loaded with a computer system, or distributed from a server over a network, for example, the Internet.

In order that it be better understood, but by way of example only the present invention will now be described with reference to the accompanying figures in which:

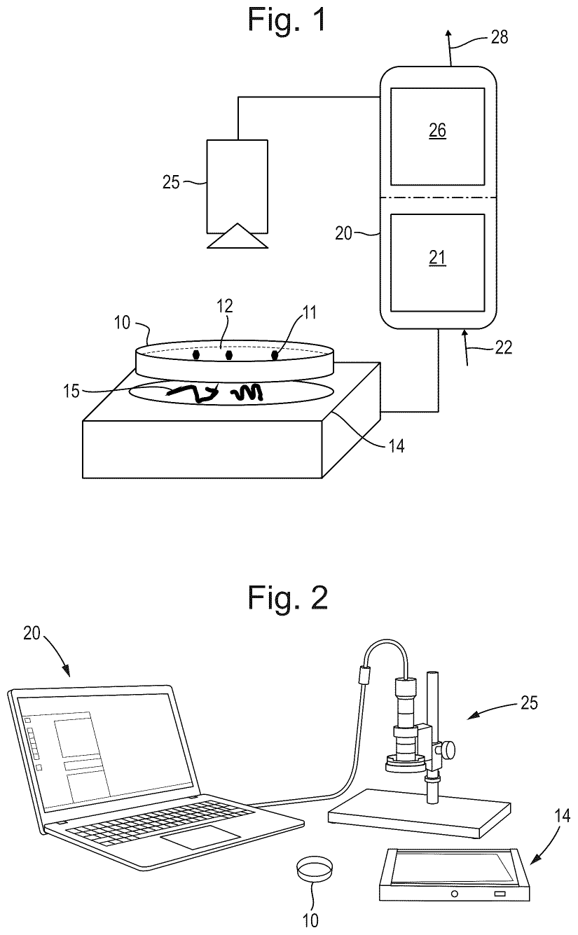

FIG. 1 is a schematic representation of one embodiment of apparatus according to the present invention;

FIG. 2 is an experimental setup of that apparatus in a broadly equivalent embodiment;

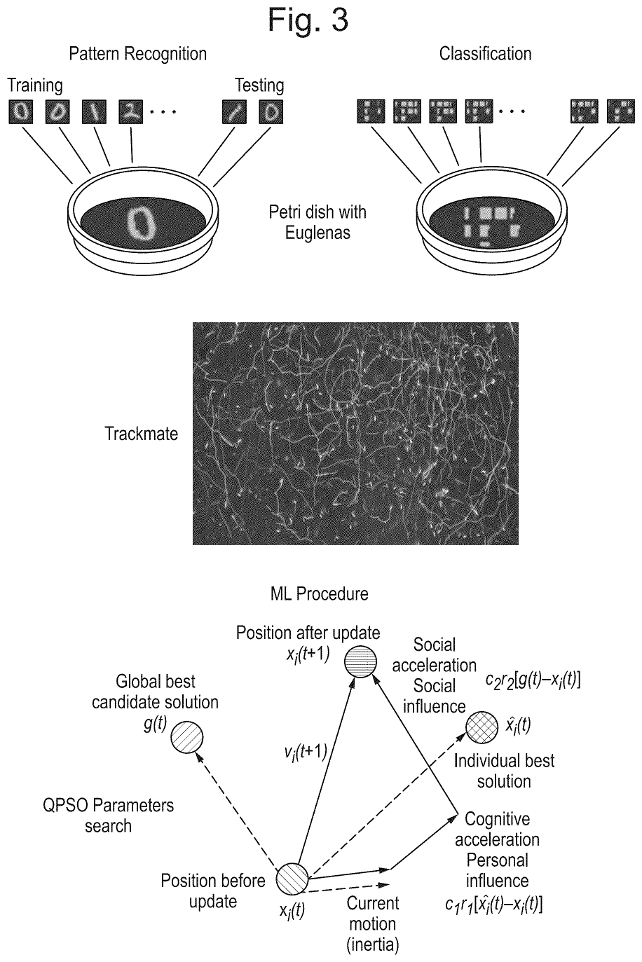

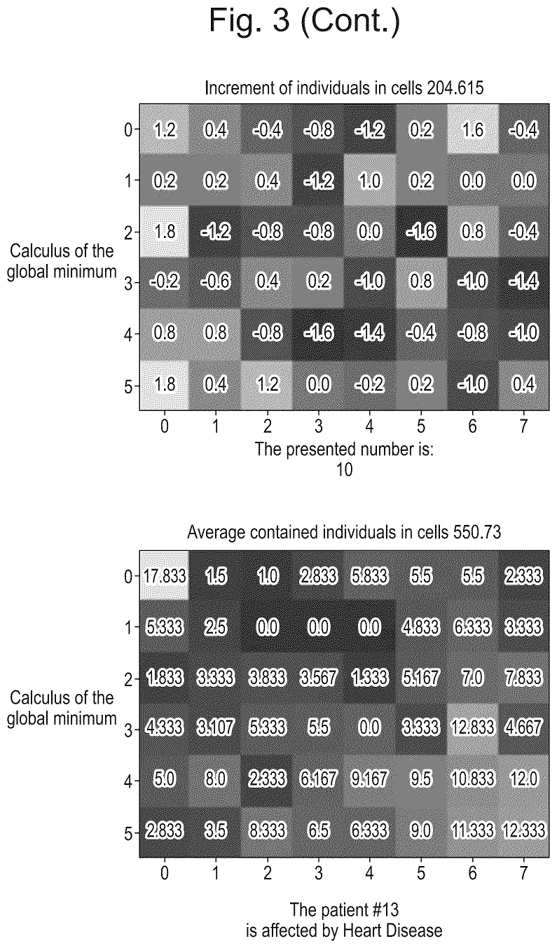

FIG. 3 is a sequential representation of stages in the operation the present apparatus and method of the invention;

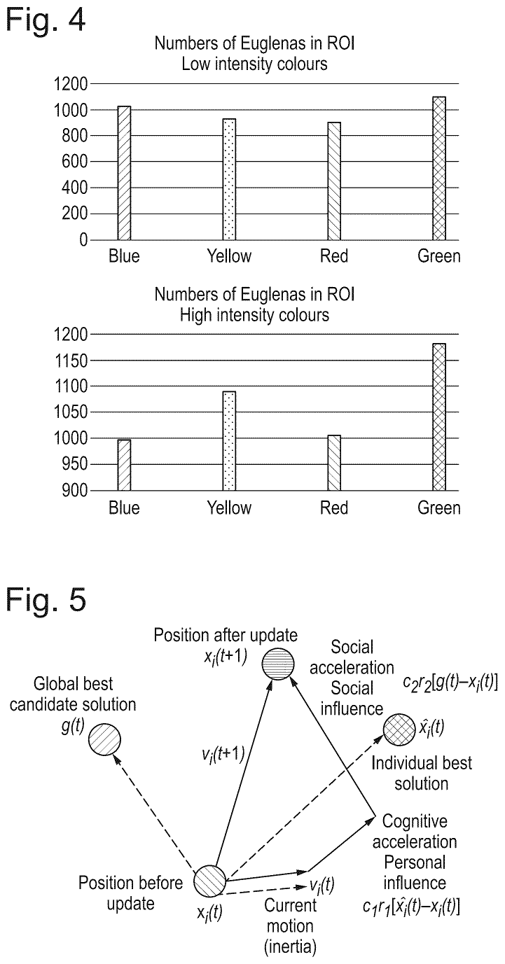

FIG. 4 shows 2 graphs of the average Euglenas responses to light of different colours at different intensities;

FIG. 5 is a graphic representation of PSO variables and PSO algorithm as related to the particle path;

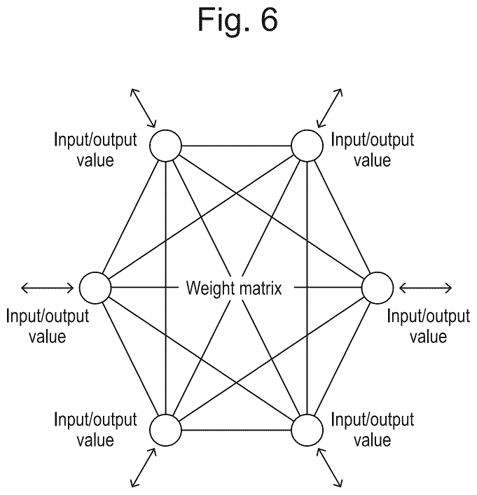

FIG. 6 is a graphic representation of the Hopfield network;

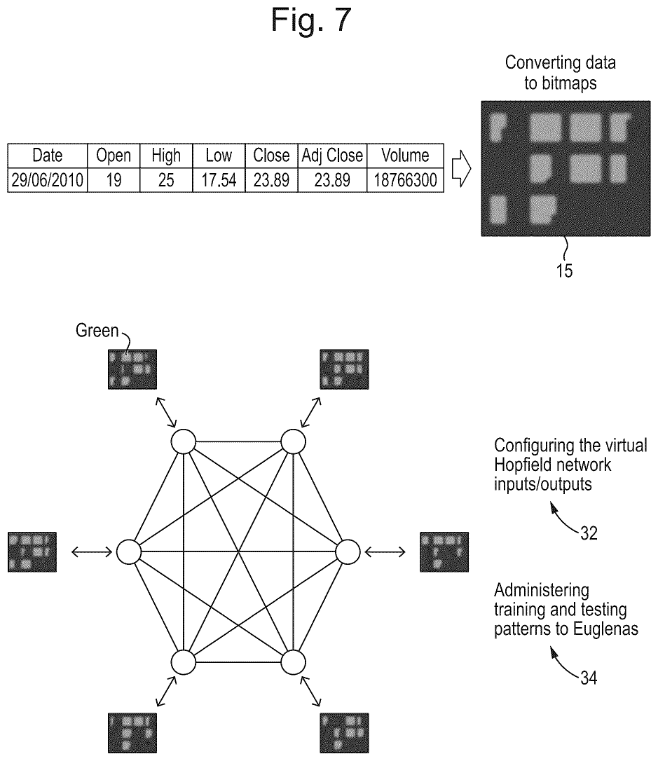

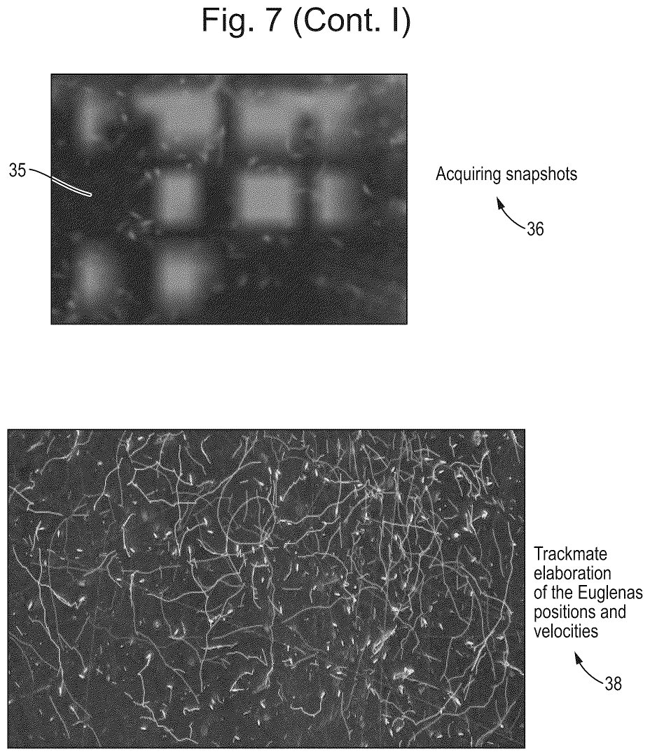

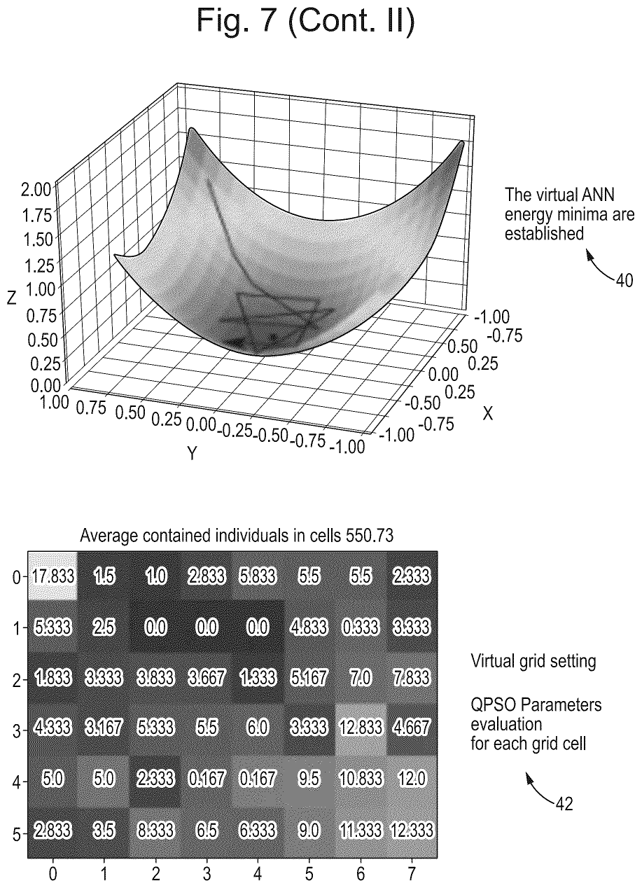

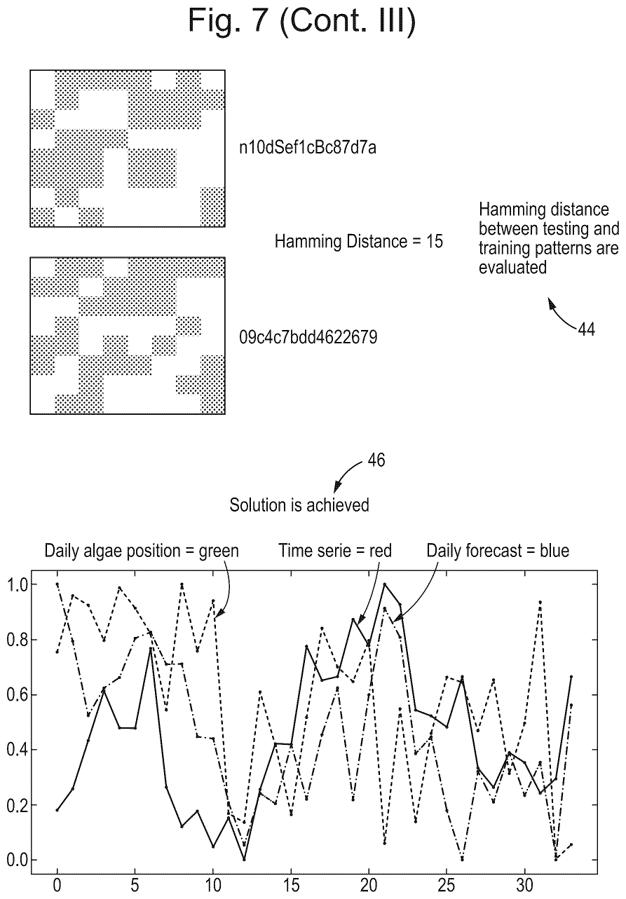

FIG. 7 is a flow chart of operational stages;

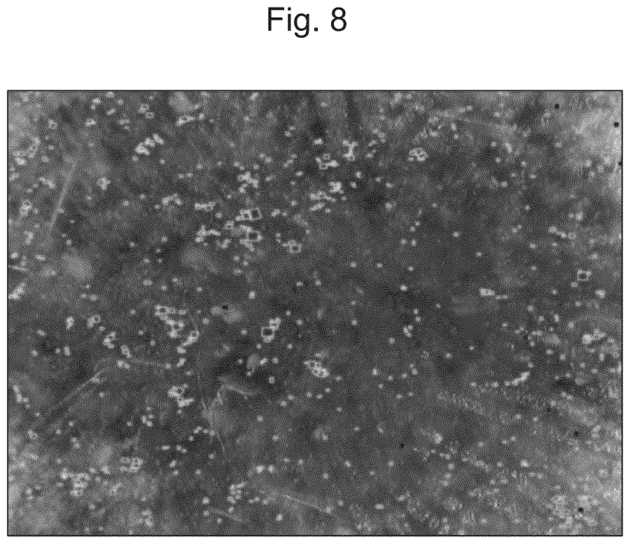

FIG. 8 shows an image of Euglenas in a working area of the receptacle before stimulation;

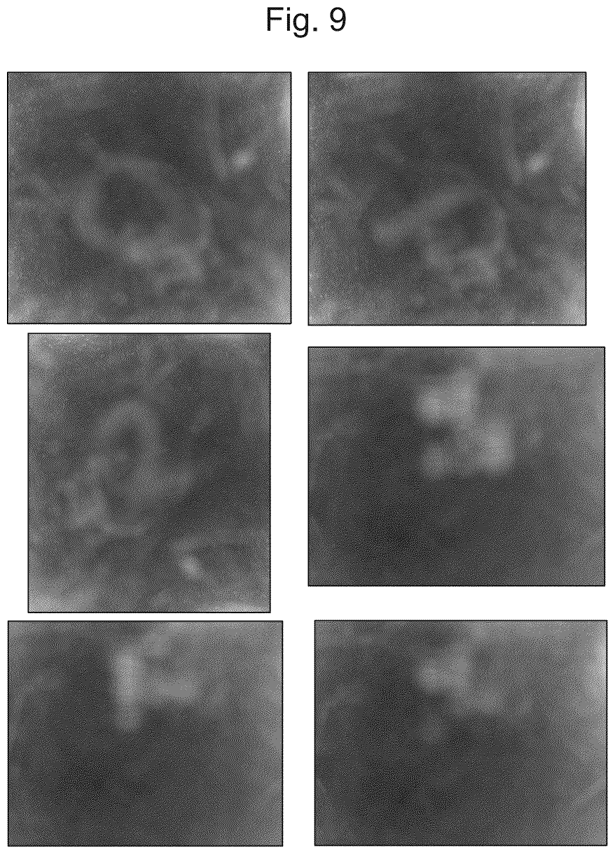

FIG. 9 shows a series of 6 bitmap images taken at different time points;

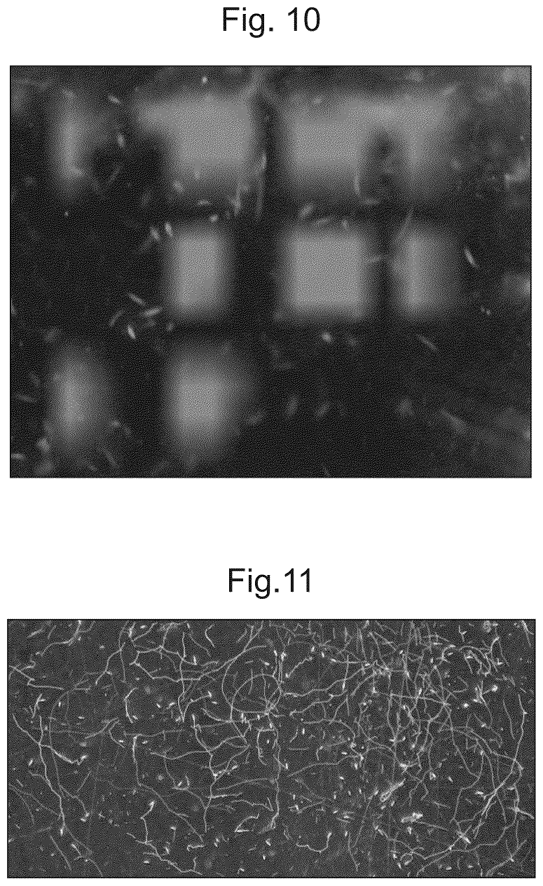

FIG. 10 shows an image of the Euglenas behaviour during the administration of a light pattern (visible in the image);

FIG. 11 shows a graphic elaboration of paths taken by individual Euglenas when stimulated; and

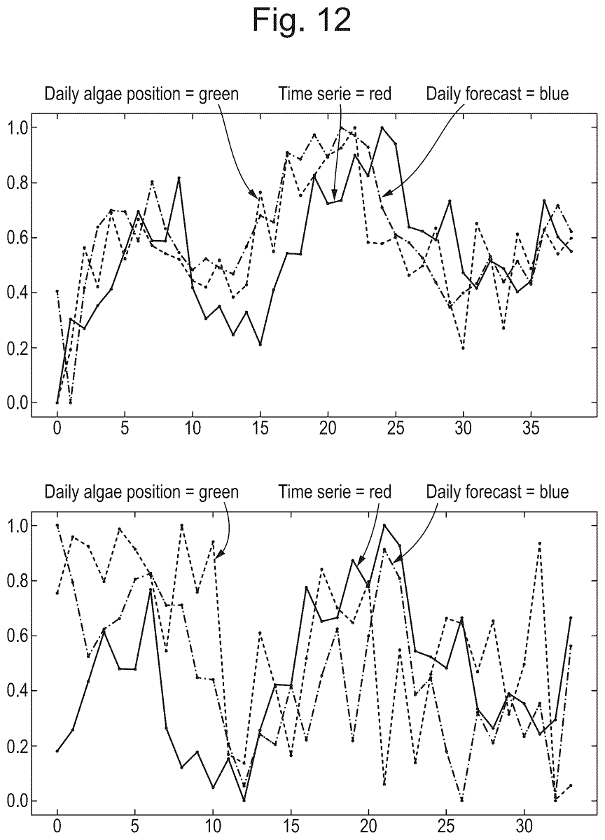

FIG. 12 shows 2 graphs of the results achieved in two different experimental sessions forecasting share values on different days.

FIG. 1 shows a schematic representation of apparatus according to the present invention. A receptacle in the form of a petri dish 10, contains a culture of microorganisms (Euglena in this example) in a growth medium 12. Several individual microorganisms are represented by shapes numbered 11, but these are not to scale nor is the number of such microorganisms accurate. The microorganisms 11 are mobile within the medium 12. Movement in the major plane of the petri dish is of most interest so the depth of growth medium 12 is less critical than the area of the dish 10.

The petri dish has a transparent lower surface and it is located on a display screen 14. The screen 14 can depict a variety of images (such as the image labelled 15) to the underside of the petri dish 10. This illumination with the image 15 stimulates the microorganisms 11 so that they move within the receptacle from a starting position before stimulation to finishing position after stimulation for a suitable time.

The nature of the image/patterns (or sequence of repeated or differing images/patterns) is controlled by a data processing unit 20 connected to the screen 14 and running stimulation control software 21. This software 21 determines the appropriate stimulation to be applied to the microorganisms 11 based on the input data (represented by arrow 22) and the desired calculation/processing step.

Before, during and after stimulation the microorganisms 11 are imaged by a monitoring system which in this embodiment includes a camera 25 focused on the area in which the microorganisms 11 are stimulated. This camera 25 may capture a series or still images at defined time points or may record a live video over the duration of stimulation. The captured image data is fed to the data processing unit 20 (potentially via an image processing system [not shown] that may be part of or separate from the data processing unit 20) which has processing software 26 that uses the data on the movement and/or final position of the microorganisms 11 to calculate output data (represented by arrow 28) that is the desired end result of the process.

The processing software 26 and stimulation control software 21 are shown as part of a single data processing unit 20. This is convenient but they may be separate units that are able to communicate as required for efficient interaction, including feedback and learning to improve accuracy and in training.

An experimental setup is depicted in FIG. 2. It consists of a data processing unit 20 in the form of a PC, a Digital Microscope 25 as the imaging system, a display 14 that transmits the light stimulations sent by the PC, and a Petri dish 10 containing the Euglena colony. This set up uses an optical CMOS sensor for the acquisition of the image timeshots, a microprocessor for the computational procedures.

The complete setup identified as suitable is as follows.
Colonies of Euglena: Euglena gracilis Klebs (1883) strain Z CCAP 1224/5Y Isolator: Pringsheim (1949) Origin: Freshwater; ditch, Cambridge, England, UK Culture: Medium EG:JM
Colonies of Chlamydomonas: Chlamydomonas Reinhardtii Dangeard (1888), CCAP 11/32A, Isolator: Smith (1945); Origin: Soil; potato field, Amherst, Massachusetts, USA Culture: Medium 3N-BBM+V
Microscope SuperEyes T Series HD Industrial Digital Table Microscope 16 Mpixel Model T006 Resolution 1920×1080 (HDMI, USB output)
Computer—ASUS LapTop A540NA-GQ260T PC, 15.6″ HD No Glare, Intel celeron N3350 1.1 GHz, Intel HD Graphics 500 shared graphics card
Light stimulator—7 inch Eyoyo E7S Full HD 1920*1200 Ultra Slim 4K/HDMI monitor
Software:
   ImageJ (open source image processing software used mainly in the field of biological research) https://imagej.net/
   Fiji (ImageJ-based environment) https://fiji.sc/
   Python custom code

The cyanobacteria strains were purchased from: Culture Collection of Algae and Protozoa, SAMS Limited, Scottish Marine institute, Scotland, United Kingdom

The culture medium: was EG:JM

EG (Euglena gracilis Medium)
Freshwater algae and protozoa

| | per litre | |
|---|---|---|
| Stock | | |
| (1) CaCl₂ stock solution: CaCl₂ | 1.0 | g |
| Medium | | |
| Sodium acetate trihydrate | 1.0 | g |
| “Lab-Lemco” powder (Oxoid L29) * | 1.0 | g |
| Tryptone (Oxoid L42) * | 2.0 | g |
| Yeast extract (Oxoid L21) * | 2.0 | g |
| CaCl₂ stock solution (1) | 10.0 | ml |

Add constituents above and make up to
1 litre with deionized water. For agar
add 15 g per litre Bacteriological Agar
(Oxold L11)*. Autoclave at 15 psi for 15 minutes.
Supply
* Unipath Ltd, Wade Road, Basingstoke, Hants RG24 0PW, UK

Culture conditions: Cyanobacteria were maintained in chemically defined medium (EG:JM) at 20° C. in non-agitated glass tubes and sub-cultured (1:10) every two weeks. A dark-light cycle of 8h/16h was used. Light lamps of about 18 W/m² consisted of a mixed cool white and warm tone.

Experimental conditions: prior to experiments, one-week old cultures (in the dark phase) were diluted 1:100 in fresh medium (to obtain about 1000 E. gracilis cells/ml) and 2 ml seeded in 35 mm Petri dishes. Cultures were kept in the light at room temperature until visualization at the microscope to promote phototaxis.

The selection of appropriate microorganisms and a phototactic response as the best response to organised stimuli resulted from an extensive bibliographic survey and several laboratory experiments observing taxis with various strains such as: E. Coli, Euglenas from different strains, Volvox, Nostoc, Chlamydomonas. In particular, phototaxis in two specific strains were found as optimal as reaching the quickest and best directional reactions.

It has been found that for these microorganisms an important issue is the wavelength (or band or wavelengths) of the light signal. The best choice of frequency and intensity was evaluated on the basis of three specific experiments in which the number of microorganisms (Euglenas and Chlamydomonas) migrating by phototaxis towards different coloured stimuli was measured, then compared. Four colours (green, blue, red, yellow) were tested with two different intensities, one twice as big as the other (as these organisms can react with attraction or repulsion from light depending on its intensity). The average of the Euglenas responses is depicted in FIG. 4. Chlamydomonas responses were quite similar and confirmed green as the colour favouring the best phototactic response in these species.

Since there were no differences in performance between Euglenas and Chlamydomonas, the Euglena microorganisms were used in subsequent experiments, as they are larger and can be more easily identified by image processing algorithms and are more safely manipulated in microbiology laboratory procedures.

FIG. 3 shows a flow-chart of the operation of the apparatus and methods of the present invention.
1. In this step, patterns (two-dimensional images in the case of an image recognition problem, or for other cases bitmaps obtained from the transformation of numerical data with the stimulation control software) are repeatedly administered to the Euglenas contained in the Petri dish in a training phase. The data of the problem to be solved are then administered in the same way (testing phase). In this testing phase it can be thought that the data are administered to a virtual ANN (Hopfield network) and the Euglenas will provide the computation system of the network.
2. The Euglenas move towards the light stimuli and software (Trackmate software—a function of Fiji which is an ImageJ-based environment) calculates the path of each microorganism.
3. The evaluation of the parameters (QPSO parameters) for the calculation of the global minimum of the ANN that represent the solution to the problem begins.
4. The area in which the Euglenas move (around 1 cm²) is superimposed on a virtual grid, and the useful parameters are calculated for each cell of the grid.
5. Once the minimum/minimums are identified, a comparison of the output with the behaviour of the Euglenas with respect to the training patterns gives the solution to the problem.

In order for the microorganisms to perform a problem-solving task, the properties of the colony as a dynamic self-organising system was used. In this way it has been found that one is able to use its behaviour as a problem solving method, because every dynamic system, if left to evolve, tends to stabilise in attractors that identify the minimum energy points of the system. This also applies to ANNs, which are dynamic systems as well.

The present example of the invention consists in building an isomorphism between the bidimensional, real-word, microorganism system, and a model of dynamic system, identifying a way to consider the microorganism system energy to be minimized and to detect the system attractors.

This method superimposes a virtual ANN on the spontaneous dynamic behaviour of microorganisms, and the search for minimum energy of the ANN leads to identification of the best solution. To this end a Hopfield network was selected as a virtual superimposed ANN.

An algorithm was developed having the Hopfield network as a model of the system behaviour, the algorithm provides input data to the microorganism in the form of stimulation and by analysing their paths identifies bidimensional or point attractors in the real-world bidimensional space.

To identify the global energy minima of the network a Swarm Intelligence algorithm was used. Swarm Intelligence is a branch of machine learning (ML) that takes its cue from the collective behaviour of many individuals of certain animal species (fish, birds, ants, bees, etc.) to solve computational problems. A Particle Swarm Optimization (PSO) algorithm was chosen as it is considered an extremely effective performance optimizer. PSO appears to be the best choice for the search of minimum to be applied to the ANN, both for its valid computational performances, and because it has been found to be particularly suitable to be applied to the collective behaviour of microorganisms.

The PSO considers the evolution of a large number of particles in a multidimensional space. Each particle represents a possible solution to the problem in question, and its movement is calculated to approach the minimum of the cost function of interest. FIG. 5 represents the PSO variables.

This algorithm is used in optimisation problems. In particular it can be applied to artificial neural network models to optimize weights by minimizing the error instead of more common and less effective algorithms (e.g. gradient descent).

The Hopfield network (a representation of which is shown in FIG. 6) is the best example of how an ANN is a dynamic system that tends to a series of stable attractors. It is a fully connected network with coincident input and output nodes and symmetrical weights, with binary or real input. The inputs are applied to all nodes simultaneously. In the learning cycle each output of a neuron is a new input for the same neuron.

One can see an input pattern as a point in the state space that, while the network iterates, moves gradually toward minimums, which represent stable states of the network. The solution occurs when the point moves to the lowest region of the basin of attraction.

The Hopfield network has not only an architecture easily configurable within the microorganism framework, but has interesting performances as an optimiser, so it lends itself to solve many important but computationally challenging problems. The equilibrium points, that represent the solutions of the dynamic system, are in fact minimum energy points, therefore suitable to represent the maximum or minimum of any function. Local minima can exist in addition to the global minimum and a good algorithm must be able to find the global minimum without being trapped in the local minima.

The Hopfield network represents the best choice as a model in conjunction with PSO, because this ANN behaves as a dynamic system itself, tending to energy minima, therefore has a behaviour isomorphic to the PSO algorithm and to the microorganisms as well. This makes it suitable to transform input data in into stimuli and to interpret the microorganism behaviour in terms of search of solutions. In other setups and applications within the framework of this patent, other models of computational intelligence may turn out to be better choices.

Like any ANN, the network can be used as classifier or approximator of any function. For example, the input data/layer can be:
An image (represented in bitmap form)
A signal (sequence of real numbers): biological signal, voice, telecommunication channels, etc.
A series of variables, one value per node: e.g. each node represents a laboratory value and/or the presence/absence of a symptom, and the output coding will be the diagnosis/classification of the patients.
The representation of a function or a problem: e.g. in the case of the classical TSP (Travelling Salesman Problem) the input neurons can represent the coordinates of the city, and the output data/layer will eventually indicate the visiting order of the city.

An interesting property of certain microorganisms such as those used herein is the ability to be sensitive to a single photon, that implies that they may exhibit quantum properties. This characteristic opens the way to the possibility to exploit quantum properties which referring to the theoretical power of Quantum computation, potentially allows one to obtain results of several orders of magnitude faster, compared to classical computational algorithms. In this regard several possibilities have been analysed, in particular:
use of microorganism as p-bit
use of quantum neural networks
theoretical models of quorum sensing through K-ion diffusion based on quantum models of cellular communication.

However, the most immediate path was taken thanks to identified PSO as suitable for the present invention such that it was possible to adopt the quantum version of this algorithm Quantum Particle Swarm Optimization (QPSO).

QPSO was introduced as a dynamic variant of PSO and was inspired by the quantum model of an atom. In this model, electron positions orbit a nucleus in a non-deterministic fashion. Thus, a proportion of particles in the QPSO algorithm are designated as “quantum” particles, and these particles do not update their positions using a velocity vector. Rather, the positions of quantum particles are sampled from a probability distribution centred within a radius (i.e., a hypersphere) around the global best position.

FIG. 7 shows a software flow-chart representing the operation of the software used. That software has been written in Python and carries out different tasks:
It configures light stimuli with intensity proportional to the input values, or that are bitmaps representing the patterns (e.g. images 15) to be recognised (step 30). In the images the patterns may be shown in green.
In configuring a virtual Hopfield network (step 32), it arranges a learning and testing procedure (step 34): it drives the pattern stimulation under the microorganism dish according to a suitable mode of training and testing: e.g. for both training phase and testing phase it may present each pattern for a suitable time (e.g. 5 seconds), interspersed with periods of darkness (for example 5 seconds).
The software acquires timeshots (36) (images at certain defined times in the process) of the microorganism and transmits them to the Trackmate software, which processes the individual path of each microorganism. (Step 38)
Since the QPSO algorithm is based on the path of the particles towards the global minimum, a tracking system (such as Trackmate—a function of Fiji which is an ImageJ-based environment) is used, which allows one to identify the position of each individual microorganism over time, combined with a counting system applied to the regions of interest (ROI). (Step 38)
Another Python module calculates the centre of gravity of the microorganisms for each timeshot and follows its trajectory over time.
On this trajectory the various parameters specific to each path are calculated, i.e. those related to the QPSO algorithm, which identifies the optimal minimum points such as those where microorganisms concentrate in attractors, where their position is fixed. (Step 40)
The software divides the field into a virtual grid of cells, one for each parameter, and for each cell the corresponding Trackmate parameters are calculated, both for training and testing patterns. Accordingly to the quantum paradigm of PSO, QPSO parameters processed for this purpose are: average number of contained individuals in a cell and the Increment of individuals within a cell. (Step 42)
Finally, the software calculates (step 44) the Hamming distance between the cells of the grids with respect to the calculated parameters, and identifies the solution for the testing pattern as the training pattern that has a minimum Hamming distance with respect to the testing pattern. The Hamming distance measures the number of substitutions needed to convert one binary string into another, or, in the present case, the minimum number of errors that may have led to the transformation of one grid into another.

A global minimum and some local minima are generally identified, and the specificity of this set of minima identifies the solution pattern (as represented in step 46).

FIG. 8 shows an image of Euglenas in the working area of the receptacle before stimulation. From this they can be counted and located—the light-coloured rectangles (which were green in the original image) show locations thereof. FIG. 9 shows 6 images of Euglenas behaviour during the administration of image bitmaps (images of the numerals 0, 1 and 2) and of data patterns converted to bitmaps. Each bitmap represents a single record with more fields. The bitmaps are in focus for the Euglenas thus they are out of focus in these images. The lighter areas appear green in the original images.

FIG. 10 shows an image of the Euglenas behaviour during the administration of a pattern. In this case the image is focused on the light pattern and the Euglenas are not highlighted.

A graphic elaboration (created by ImageJ elaboration) of the path of individual Euglenas is shown in FIG. 11. From this the direction and speed of travel as well as the start and finish positions can be visualised.

The experimentation took place by applying the system thus configured to three different types of problems and data namely:
a. pattern recognition
b. data analysis
c. time series forecast.

For the pattern recognition experiment (a) a series of 5 numbers were used, digital versions of handwritten numbers.

The experiment consists in the repeated administration of training patterns, in the form of pure green bitmaps, consisting of different versions of the same number, interspersed with a few seconds of darkness, followed by the recording of the timeshots of the microorganisms' movement for a number of seconds. The optimal number of seconds was experimentally determined to work out the number of seconds needed by the micro-organisms, once stimulated by the light patterns, to cross the field and stabilize, before diverging driven by spontaneous movements. This training phase is followed by the testing phase and the administration of test patterns, interspersed with a few seconds of darkness, which are the patterns to be determined.

Once the sequence of trajectories of the microorganisms has been recorded, it is necessary to compare the final configuration obtained after the administration of the testing patterns with the configurations of the training phase. For this purpose the software follows the flowchart in FIG. 7, and as mentioned above:
it calculates the barycentre of the microorganisms cloud for each timeshot and its trajectory over time is followed.
with Trackmate it calculates the path of the microorganisms and the various parameters specific to each path, in particular those related to the QPSO algorithm, which identifies the optimal minimum points such as those in which the microorganisms concentrate in attractors, where they are stationary.
the field is divided into a virtual grid of cells (it was identified experimentally that an 8×6 size gave the best results), and for each cell the corresponding Trackmate parameters are calculated: both for training and testing patterns.
finally, the Hamming distance between the grids is calculated with respect to the calculated parameters, and identifies a solution for the testing pattern from the training pattern that has minimum Hamming distance with respect to the testing pattern.

A test experiment has been conducted by identifying with very satisfactory percentages the testing pattern among a choice of 24 training patterns of type 0, 1, 2, 3 and 4.

The same experiment was then repeated using biomedical data to identify cardiovascular risk (b) (data analysis). It was possible correctly to classify the cardiological risk into two categories, but also to label, through comparison with the training data, the patient's belonging to one group rather than another.

Another experiment (c) was carried out with time series from the Stock market (TESLA Stock data from 2010 to 2020). The aim of the experiment was to predict the price on the following day.

The steps of the experiment are described in the flowchart of FIG. 7 and are similar to those in (a) and (b), but using all the known values in succession and going to predict the next value according to the spontaneous evolution of the microorganisms at the end of the administration of the time series values. The data used were:
Opening price
Highest price that day
Lowest price that day
Closing price
Adjusted closing price, taking splits etc into account
Trading volume

The bitmaps related to the values of the time series are presented in succession, each three times followed by a short dark period.

The centre of gravity of the Euglenas positions is evaluated as the bitmaps advance and analysed with Trackmate. As a predictive value, the barycentre position of the Euglenas in the last dark timeshot is taken, i.e. allowing the Euglenas to evolve freely for a short period after the previous bitmap training.

Finally the new barycentre position is compared with those of the known input positions to determine the correspondence with a numerical value. The results achieved are evident in the graphs of FIG. 12 and concern two different experimental sessions forecasting values in different days, where the red line represents the historical times series, the green line represents the path of the Euglena stimulated by the time series values and finally, the blue line represents the forecast time series generated by the hybrid system computation. As can be seen the system generates a very good forecasting fit, showing the same good performance as in the case of the two above mentioned experiments.

The inventors have developed, within the scope of invention, an instance of a computational problem-solving method based on ANNs with QPSO optimisation. They have invented apparatus, method and software capable of processing the reaction of microorganisms in response to appropriate stimuli. In particular, they have invented a bioelectronic technology (software+hardware+microorganism culture) capable of processing information, as well as software suitable for processing the response of colonies of microorganisms to solve problems. The described specific instance of the invention is only an example of a more general method, based on the interaction of software, hardware and microorganism cultures, cooperating in order to perform computations and problem solving.

The aspects of the present invention can make contributions in a number of areas, replacing or supplementing silicon-based computational methods by providing a low-cost microscopic system of computational intelligence.

An important aspect of the invention is that ancillary digital computations are limited and can be realized on simple computational apparatus, as in all the above-described integrative computation algorithms, carried out on the images of microorganisms, that have linear computational load and insensitive computational time. Realized so far in sequence, as described above, they can be engineered in order to be automated and transferred to a super-thin printed circuit board mounted above the microorganism plate. Miniaturisation can take place with technologies already available.

As a result, the present invention can replace the use of computers with a computational intelligence system of minimal size. The ancillary part of the computation takes place on electronic circuitry, while the part that depends on data and intelligence is constituted by the microorganism system.

Uses of the present invention can be identified in the processing of information in an analogue-analogue context where a direct interface between materials of different nature, organic and inorganic, and computational systems is required. Microorganism plates can be used for industrial sensors, put in contact with air or liquid samples (e.g. for the evaluation of environmental pollution), also for continuous monitoring where an interface between biological and computational systems is required: e.g. monitoring systems applied to the skin. When applied to the skin the present invention could in a few seconds evaluate parameters related to pathologies, emotions, states of consciousness, and aptitudes. This could have varied application including for health assessments, commercial assessments or lie detection.

The apparatus of the present invention could be developed such that blood or serum samples or histological samples may be introduced in such a way to lead to stimulation of the microorganisms and thus the invention perform complex analysis thereof in seconds.

The same methods could also be used in industrial applications, for example, for replacing digital sensors and the corresponding detection procedures with microorganism based systems, or for control systems coupled with industrial process.

Moreover, the apparatus of the present invention could be used for big data analyses, on a variety of different application areas.

An advantage of using microorganism systems of the present invention is that they can be made cost effectively and the same extremely inexpensive set of microorganisms can be used for different analyses, replacing expensive sets of analysis machines.

In a digital context, the present invention can support robotic and bionic systems by performing local artificial intelligence computations without the use of supporting computers.

It is possible that in due course the microorganisms can be optimized with regard to response to stimulus (e.g. phototactic function), both by acting on a genetic level and through the laboratory technique of subsequent selection of the best individuals.

The use of the QPSO algorithm allows a natural application of the procedure to any optimization problem, both as an end in itself (for example in TSP-like problems) and as a method of error minimisation in any problem solvable by an artificial neural network. Supporting computational algorithms, starting from microorganism counting algorithms, can be replaced over time by other solutions developed.

In addition to the computational problems described, numerical computational problems or complex problems requiring non-deterministic or multi-processing procedures can also be addressed. For this purpose it would be possible to implement algorithms other than those considered. A possible solution is to adopt calculation methods that refer to known algorithms, such as Petri nets, planning trees, belief networks, decision trees, etc. It is possible to write an algorithm of any nature, even numerical calculation, or with uncertainty management, and translate it into an appropriate network executable by microorganisms. For example, in Petri nets each token will be represented by the ignition of a point on the plane, and the microorganisms will move towards that point, making the network evolve. In the case of computation of processes, even complex ones, where the choice can be represented in a binary way, the microorganisms will proceed along the network leading to the result.

If one thinks about numerical algorithms, one needs a method of quantification. As seen, in the example the light intensity is varied in a controlled way: e.g. microorganisms are attracted to a point (the place of the network) with intensity 3.5 and another with intensity 7.8. Then the network evolves, and the necessary arithmetic operations are done externally attributing the externally calculated luminous intensity to the various places. In other words, the logical behaviour is left to the microorganisms and algebraic calculations are made externally. This allows precision to be maintained regardless of the stochastic behaviour of the microorganisms.

### REFERENCES

Berg, H. C. & Brown, D. A. Chemotaxis in Escherichia coli analysed by three-dimensional tracking. Nature 239, 500-504 (1972).
Amanda Galante, Susanne Wisen, Devaki Bhaya, and Doron Levy, Modeling Local Interactions during the Motion of Cyanobacteria. J Theor Biol. 2012 Sep. 21; 309: 147-158.
Nikita Vladimirov, Victor Sourjik, Chemotaxis: how bacteria use memory. Biol Chem. 2009 November; 390(11):1097-1104.
M. Mehdi Salek, Francesco Carrara, Vicente Fernandez, Jeffrey S. Guasto & Roman Stocker, Bacterial chemotaxis in a microfluidic T-maze reveals strong phenotypic heterogeneity in chemotactic sensitivity. Nature Communications volume 10, 1877 (2019).
Tae-Yun Kang, David Ellison, Sung Hoon Lee, Andrew J. Ewald, Andre Levchenko, 3D Analysis of Multi-cellular Responses to Chemoattractant Gradients. J of Visualized Experiments, May 2019, 147, e59226
Khraibi, Y. Li, Y Gu, H, Wang, Z. Liu, B. Song, T. Yin, Electric Pulses Can Influence Galvanotaxis of Dictyostelium discoideum. BioMed Research International, Vol. 2018, 2534625.
Nils Schuergers et al., Cyanobacteria use micro-optics to sense light direction. eLife 2016; 5:e12620.
Rosanna Man Wah Chau, Devaki Bhaya, b Kerwyn Casey Huanga, Emergent Phototactic Responses of Cyanobacteria under Complex Light Regimes. Mbio American Society for Microbiology, March/April 2017 Volume 8 Issue 2 e02330-16.
Rosanna Man Wah Chau, Tristan Ursell, Shuo Wang, Kerwyn Casey Huang, and Devaki Bhaya, Maintenance of Motility Bias during Cyanobacterial Phototaxis. Biophysical Journal, Volume 18 Apr. 2015, 1623-1632.
David Colliaux, Lia Giraud, Claude Yepremian, Pierre Bessiere, Jacques Droulez (2015) Probing models of minima swimming vehicules in vivo with microalgae phototaxis. Proceedings of the European Conference on Artificial Life 2015, p. 447.
P. Varuni, Shakti N. Menon & Gautam I. Menon, Phototaxis as a Collective Phenomenon in Cyanobacterial Colonies. Scientific Reports (2017) 7:17799.
Levy D, Requeijo T. Stochastic models for phototaxis. Bulletin of Mathematical Biology, 22 Jul. 2008, 70(6):1684-1706.
Donat-P Haeder, Simulation of Phototaxis in the Flagellate Euglena gracilis. Journal of Biological Physics 19: 95-108, 1993. KluwerAcademic.
Wolken J J. Euglena: the photoreceptor system for phototaxis. J Protozool. 1977 November; 24(4):518-22.
B. Diehn and G. Tollin, Phototaxis in Euglena—Physical Factors Determining the Rate of Phototactic Response, Photochemistry and Photobiology, Volume5, Issue7, 1966
K. E. Bound & G. Tollin, Phototactic Response of Euglena gracilis to Polarized Light, Nature volume 216, pages 1042-1044, 1967.
Häder D P., Ntefidou M., Iseki M., Watanabe M. (2005) Phototaxis Photoreceptor in Euglena gracilis. In: Wada M., Shimazaki K., lino M. (eds) Light Sensing in Plants. Springer, Tokyo. https://doi.org/10.1007/4-431-27092-2_25
B. Diehn, Phototaxis in Euglena 1: Physiological Basis of Photoreception and Tactic Orientation. In: Behaviour of Micro-organisms. Springer, Boston, 1973.
Hirschberg, R. & Stavis, R. Phototaxis mutants of Chlamydomonas reinhardtii. J. Bacteriol. 129, 803-808 (1977).
Jonathan A. Hollm, Ranjan P. Kahn, Ellisha N, Marongelli and William H. Guilford, Laser Trap Characterization and Modeling of Phototaxis in Chlamydomonas reinhardtii. Cellular and Molecular Bioengineering, Vol. 2, No. 2, June 2009, pp. 244-254
R. Rosen, “Dynamical System Theory in Biology,” in: Stability Theory and its applications, vol. 1, Wiley Interscience Series on Biomedical Engineering., 1970.
Maren, D. Jones, “Neural Networks for Spatio-temporal Pattern Recognition,” in: Handbook of Neural Computation Applications, Maren A. J., Harston C. T., Pap R. M. eds, Academic Press, 1991.
K. S. Narendra, K. Parthasarathy, Neural networks and dynamical systems, International Journal of Approximate Reasoning, Volume 6, Issue 2, 1992, pp 109-131
B. Cessac, A view of Neural Networks as dynamical systems, International Journal of Bifurcation and Chaos 20(6), 2009
R. Pizzi, M. Musumeci, Artificial Neural Networks, Dynamical Systems and Self-Organization, Createspace Pub, 2017.
L. Deneubourg, S. Goss, Collective patterns and decision making, Ethol. Ecol. Evol. 1 (1989) 295-311.
E. Bonabeau, M. Dorigo, G. Theraulaz, Swarm Intelligence: From Natural to Artificial Systems, Oxford University Press, Oxford, 1999.
M. Dorigo, G. Di Caro, L. M. Gambardella, Ant algorithms for discrete optimization, Artificial Life (2) (1999) 137-172.
Beni, G., Wang, J. Swarm Intelligence in Cellular Robotic Systems, Proceed. NATO Advanced Workshop on Robots and Biological Systems, 1993, Springer Berlin, pp. 703-712.
A. Czirók, T Vicsek, Collective behavior of interacting self-propelled particles, Physica A., 2006, 281(1): 17-29
Solé R, Rodriguez-Amor D, Duran-Nebreda S, Conde-Pueyo N, Carbonell-Ballestero M, Montanez R, Synthetic Collective Intelligence, BioSystems, 2016, 148, 47-61
H. Jeckel, E. Jelli, R. Hartmann, P. K. Singh, R. Mok, J. F. Totz, L. Vidakovic, B. Eckhardt, J. Dunkel, and K. Drescher, Learning the space-time phase diagram of bacterial swarm expansion. PNAS Jan. 29, 2019, 116 (5) 1489-1494.
Kennedy J, Eberhart R. Particle swarm optimization. Proc IEEE Int Conf Neural Netw 1995; 4: 1942-1948
Mohd Nadhir Ab Wahab, Samia Nefti-Meziani, Adham Atyabi. A Comprehensive Review of Swarm Optimization Algorithms. PLoS ONE 10(5) e0122827, May 18, 2015.
Banks, Alec; Vincent, Jonathan; Anyakoha, Chukwudi, A review of particle swarm optimization. Part I: background and development. Natural Computing. 6 (4), 2007, 467-484.
Yechuang Wang, Zhihua Cui and Wuchao Li, A Novel Coupling Algorithm Based on Glowworm Swarm Optimization and Bacterial Foraging Algorithm for Solving Multi-Objective Optimization Problems. Algorithms 2019, 12(3), 61.
J. J. Hopfield, Neural networks and physical systems with emergent collective computational abilities. Proc. Natl Acad. Sci. 79, 2554-2558 (1982).
J. J. Hopfield, “Neural Networks and Physical Systems with Emergent Collective Computational Abilities,” Proc. Nat. Acad. Sci USA, no. 81, 1984.
Cai, F., Kumar, S., Van Vaerenbergh, T. et al. Power-efficient combinatorial optimization using intrinsic noise in memristor Hopfield neural networks. Nat Electron 3, 2020, 409-418.
Sarangam Majumdar and Sukla Pal, Bacterial intelligence: imitation games, time-sharing, and long-range quantum coherence. J Cell Commun Signal. 2017 September; 11(3): 281-284.
David Coles, et al., A Nanophotonic Structure Containing Living Photosynthetic Bacteria. small J (2017) 13, 1701777, Wiley—VCH Verlag.
C Marletto, D M Coles, T Farrow and V Vedral, Entanglement between living bacteria and quantized light witnessed by Rabi splitting. Journal of Physics Communications, Volume 2, Number 10.
Jonathan O'Callaghan, Schrödinger's Bacterium Could Be a Quantum Biology Milestone. Scientific American Oct. 29, 2018.
Sarovar, M., Ishizaki, A., Fleming, G. et al., Quantum entanglement in photosynthetic light-harvesting complexes. Nature Phys 6, 2010, 462-467.
Pengfei Jia, Shukai Duan and Jia Yan, An Enhanced Quantum-Behaved Particle Swarm Optimization Based on a Novel Computing Way of Local Attractor. Information 6, 2015, 633-649.
K. R. Harrison, B. M. Ombuki-Berman and A. P. Engelbrecht, “A radius-free quantum particle swarm optimization technique for dynamic optimization problems,” 2016 IEEE Congress on Evolutionary Computation (CEC), Vancouver, B C, 2016, pp. 578-585, doi: 10.1109/CEC.2016.7743845.
Z. Chai, J. Sun, R. Cai and W. Xu, “Implementing Quantum-Behaved Particle Swarm Optimization Algorithm in FPGA for Embedded Real-Time Applications,” 2009 Fourth International Conference on Computer Sciences and Convergence Information Technology, Seoul, 2009, pp. 886-890, doi: 10.1109/ICCIT.2009.21.
M. Norouzi, D. J. Fleet, R. R. Salakhutdinov, Hamming Distance Metric Learning, Proc. Advances in Neural Information Processing Systems 25 (NIPS 2012)
Bookstein, V. A. Kulyukin, & T. Raita, Generalized Hamming Distance. Information Retrieval 5, 353-375 (2002).

1. Apparatus for implementing a computational system comprising:
a colony of live microorganisms;
a receptacle in which the microorganisms are able to be stimulated to cause a reaction or to modify their physiology or behaviour;
monitoring apparatus to track the reaction or changes in the physiology or the behaviour of the microorganisms in response to stimulation; and
processing circuitry to make calculations, including machine learning, based on or coupled with the reaction of the microorganisms, or their physiological or behavioural changes.

2. Apparatus as claimed in claim 1, which further includes stimulation apparatus to stimulate the microorganisms within the receptacle.

3. Apparatus as claimed in claim 1, wherein the microorganisms are selected from bacteria, cyanobacteria, single cell flagellates, viruses, archaea, protists, and unicellular fungi.

4. Apparatus as claimed in claim 1, wherein the microorganisms are from the genus Euglena.

5. Apparatus as claimed in claim 1, wherein the receptacle is a petri-dish or other substantially flat plate on or in which the microorganisms can react or be otherwise modified.

6. Apparatus as claimed in claim 1, wherein the stimulus is in the form of light.

7. Apparatus as claimed in claim 6 wherein the light stimulus can vary, in one or more of the following characteristics: light intensity, wavelength, pattern and duration.

8. Apparatus as claimed in claim 2, wherein the stimulation means comprises a display screen located adjacent the microorganisms.

9. Apparatus as claimed in claim 1 in which the monitoring apparatus includes one or more of a microscope, camera and image processing software to track reaction or modification of the microorganisms.

10. A method of implementing a computational system comprising:
stimulating a colony of live microorganisms with a stimulus whose pattern is derived from input data, or allowing the stimulation of a colony of live microorganisms with an external stimulus;
monitoring the reaction or modification of the microorganisms in response to that stimulation; and
deriving output data based on the reaction or modification.

11. The method as claimed in claim 10, in which the calculations are carried out on processing circuitry to generate the output data driven by the computations of the microorganisms, intrinsic in their reactions or modifications.

12. The method of claim 10 in which the method involves particle swarm optimisation.

13. The method as claimed in claim 10 in which the stimulation involves providing an input pattern of stimulation to the microorganisms for a defined time period.

14. The method as claimed in claim 10 in which monitoring includes visual location and tracking of the microorganisms prior to, during and after stimulation.

15. The method as claimed in claim 10 in which the microorganisms are located in a receptacle that maintains their viability and permits their reaction, behavioural change or physiological change in response to stimulation as well as allowing monitoring of their reaction, behavioural change or physiological change.

16. The method as claimed in claim 10 in which there is a training phase during which output data derived from reaction or modification of the microorganisms in response to the stimulation is gathered and may be used to adjust the stimulation pattern to improve the accuracy of output data in subsequent cycles of stimulation.

17. A data processing system configured to implement a method as claimed in claim 10, the system being configured to derive the stimulus from input data and/or to calculate the output data based on the reaction or modification.

18. A computer program comprising computer software code for implementing a method as claimed in claim 10, the code being configured to derive the stimulus from input data and/or to calculate the output data based on the reaction or modification of the microorganisms when the program is run on a data processing system.
